(19) United States
(12) Patent Application Publication
(10) Pub. No.: US 2025/0312602 A1
(43) Pub. Date: Oct. 9, 2025

(54) Treating Orthostatic Intolerance Conditions Using Spinal Cord Stimulation

(71) Applicant: Boston Scientific Neuromodulation Corporation, Valencia, CA (US)

(72) Inventors: Ismael Huertas Fernandez, Seville (ES); Cristian Rízea, Madrid (ES)

(21) Appl. No.: 18/991,320

(22) Filed: Dec. 20, 2024

Related U.S. Application Data
(60) Provisional application No. 63/575,970, filed on Apr. 8, 2024.

(51) Int. Cl. A61N 1/36; A61N 1/05; A61N 1/372

(52) U.S. Cl. CPC A61N 1/36135; A61N 1/0551; A61N 1/36057; A61N 1/36062; A61N 1/36114; A61N 1/37247

(57) ABSTRACT

Techniques are disclosed using a Spinal Cord Stimulation (SCS) Implantable Pulse Generator (IPG) to treat Postural Orthostatic Tachycardia Syndrome (POTS) or other orthostatic intolerance conditions. When treating POTS, SCS is preferably applied to recruit splanchnic nerves, thus modulating recruitment of blood volume to the organs in a patient's splanchnic bed. When employed in this context, non-destructive stimulation is preferably provided to the dorsal horn in the patient's spinal column, which modulates the intermediolateral nucleus (IML) that includes sympathetic pre-ganglionic neurons (SPNs) that ultimately affect the sympathetic nervous system of splanchnic nerves innervating the splanchnic bed. Use of the IPG can be supplemented by use of one or more sensors, such as a heart rate, blood pressure, and/or patient position sensor, which can be used to assess the effectiveness of the stimulation in treating POTS, and to provide closed loop control of the IPG.

### CROSS REFERENCE TO RELATED APPLICATIONS

This is a non-provisional application of U.S. Provisional Patent Application Ser. No. 63/575,970, filed Apr. 8, 2024 which is incorporated herein by reference, and to which priority is claimed.

### FIELD OF THE INVENTION

This application relates to Implantable Spinal Cord Stimulator (SCS) devices, and more specifically to use of such devices in treating Postural Orthostatic Tachycardia Syndrome (POTS) and other orthostatic intolerance conditions.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system. However, the present invention may find applicability with any stimulator device system.

A stimulator system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads 15 that form an electrode array 17. For example, one or more percutaneous leads 15 can be used having ring-shaped or split-ring electrodes 16 carried on a flexible body 18. In another example not illustrated, a paddle lead can provide an electrode array 17 positioned on one of its generally flat surfaces. Lead wires 20 within the leads are coupled to the electrodes 16 and to proximal contacts 22 insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins to stimulation circuitry 28 (FIG. 3) within the case 12.

In the illustrated IPG 10, there are sixteen electrodes (E1-E16), split between two percutaneous leads 15, and thus the header 23 may include two lead connectors 24. However, the type and number of leads, and the number of electrodes, and the number of lead connectors in an IPG is application specific and therefore can vary. The conductive case 12, or some conductive portion of the case, can also comprise an electrode (Ec). In an SCS application, the electrode lead(s) 15 are typically implanted in the spinal column inside the patient's vertebrae and proximate to the dura in a patient's spinal cord, preferably spanning left and right of the patient's spinal column. The proximal contacts 22 are tunneled through the patient's tissue to a distant location such as the buttocks where the IPG case 12 is implanted, at which point they are coupled to the lead connectors 24. SCS therapy is traditionally used to relieve symptoms such as chronic back pain. IPG 10 as described should be understood as including non-implantable External Trial Stimulators (ETSs), which mimic operation of the IPG 10 during trials periods when electrode array 17 has been implanted in the patient but the IPG 10 has not. See, e.g., U.S. Pat. No. 9,259,574 (disclosing an ETS).

IPG 10 can include an antenna 26a allowing it to communicate bi-directionally with a number of external devices discussed subsequently. Antenna 26a as shown comprises a conductive coil within the case 12, although this coil antenna can also appear in the header 23. When antenna 26a is configured as a coil, communication with external devices preferably occurs using near-field magnetic induction. IPG 10 may also include a Radio-Frequency (RF) antenna 26b. In FIG. 1, RF antenna 26b is shown within the header 23, but it may also be within the case 12. RF antenna 26b may comprise a patch, slot, or wire, and may operate as a monopole or dipole. RF antenna 26b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases (30), as shown in the example of FIG. 2. Stimulation parameters typically include amplitude (current I, although a voltage amplitude V can also be used); frequency (F); pulse width (PW); the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2, electrode El has been selected as an anode (during its first phase 30a), and thus provides pulses which source a positive current of amplitude +I to the tissue. Electrode E2 has been concurrently selected as a cathode (again during first phase 30a), and thus provides pulses which sink a corresponding negative current of amplitude −I from the tissue. This is an example of bipolar stimulation, in which the electrode array 17 includes one anode pole and one cathode pole, as discussed later with respect to FIG. 5A. Stimulation provided by the IPG 10 can also be monopolar, with the electrode array 17 programmed with a single pole of a given polarity (e.g., a cathode pole), and with the conductive case electrode Ec acting as a return (e.g., an anode pole). Multipolar (e.g., tripolar) stimulation can also be used, with the electrode array 17 having three or more poles. Note that more than one electrode in the electrode array may be active to form a pole in the electrode array, as discussed and shown further below. See also U.S. Pat. No. 10,881,859, which is incorporated herein by reference in its entirety.

IPG 10 as mentioned includes stimulation circuitry 28 to form prescribed stimulation at a patient's tissue, and FIG. 3 shows an example of such circuitry. The stimulation circuitry 28 shown includes one or more current source circuits and one or more current sink circuits. The sources and sinks can comprise Digital-to-Analog converters (DACs), and may be referred to as PDACs and NDACs in accordance with the Positive (sourced, anodic) and Negative (sunk, cathodic) currents they respectively issue. In the example shown, a NDACi/PDACi pair is dedicated (hardwired) to a particular electrode node ei 39. Each electrode node ei 39 is associated with an electrode Ei 16 via a DC-blocking capacitor Ci 38, for the reasons explained below. The stimulation circuitry 28 in this example also supports selection of the conductive case 12 as an electrode (Ec 12), which case electrode is typically selected for monopolar stimulation as explained above. PDACs and NDACs can also comprise voltage sources.

Proper control of the PDACs and NDACs allows any of the electrodes 16 to act as anodes or cathodes to create a current through a patient's tissue, R, hopefully with good therapeutic effect. Consistent with the example provided in FIG. 2, FIG. 3 shows operation during the first phase 30a in which electrode El has been selected as an anode electrode to source current I to the tissue R and E2 has been selected as a cathode electrode to sink current I from the tissue. Thus PDAC1 and NDAC2 are digitally programmed (Ip1, In2) to produce the desired current, I, with the correct timing (e.g., in accordance with the prescribed frequency and pulse widths). As mentioned above, more than one anode electrode and more than one cathode electrode may be selected at one time, and thus current can flow through the tissue R between two or more of the electrodes 16. Other stimulation circuitries 28 can also be used in the IPG 10, including ones that include switching matrices between the electrode nodes ei 39 and the N/PDACs. See, e.g., U.S. Pat. Nos. 6,181,969, 8,606,362, 8,620,436, 11,040,192, and 10,912,942. Much of the stimulation circuitry 28 of FIG. 3, including the PDACs and NDACs, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain, or be coupled with, other circuitry useful in the IPG 10, such as a microcontroller, telemetry circuitry (for interfacing off chip with telemetry antennas 26a and/or 26b), circuitry for generating the compliance voltage VH which powers the stimulation circuitry 28, various measurement circuits, etc. Collectively, such circuitry comprises control circuitry in the IPG 10.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861. While useful, DC-blocking capacitors 38 are not strictly required in all IPG designs and applications.

Referring again to FIG. 2, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. During the first pulse phase 30a (e.g., PDAC1 and NDAC2 are activated), charge will (primarily) build up across the DC-blockings capacitors (e.g., C1 and C2) associated with the electrodes (e.g., E1 and E2) used to produce the current. During the second pulse phase 30b, when the polarity of the current I is reversed at the selected electrodes E1 and E2 (e.g., PDAC2 and NDAC1 are activated), the stored charge on capacitors C1 and C2 is recovered.

Charge recovery using phases 30a and 30b is said to be “active” because the P/NDACs in stimulation circuitry 28 actively drive a current, in particular during second phase 30b to recover charge stored after the first phase 30a. However, such active charge recovery may not be perfect, and some residual charge may be present in capacitive elements in the current path even after phase 30b is completed. Accordingly, the stimulation circuitry 28 can also provide for passive charge recovery. Passive charge recovery is implemented using passive charge recovery switches 41 as shown in FIG. 3. These switches 41 when selected via assertion of control signals Xi couple each electrode node ei to a particular circuit node (shown here as the battery voltage Vbat, although another DC node could be used as well). As explained in U.S. Pat. Nos. 10,716,937 and 10,792,491, this allows any stored charge to be recovered through the patient's tissue, R. Control signals Xi are usually asserted to cause passive charge recovery after each pulse (e.g., after phase 30b) during periods 30c shown in FIG. 2, and are at least asserted in the previously active current paths: that is, at least X1 and X2 would be asserted in the example of FIG. 2 (although all control signals Xi could also be asserted). Because passive charge recovery involves capacitive discharge through the resistance R of the patient's tissue, such discharge manifests as an exponential decay in current, although this is not shown in FIG. 2. Passive charge recovery during period 30c may be followed by a quiet period 30d during which no active current is driven by the DAC circuitry, and none of the passive recovery switches 41 are closed. This quiet period 30d may last until the next pulse is actively produced (e.g., phase 30a). Like the particulars of pulse phases 30a and 30b, the occurrence of passive charge recovery (30c) and any quiet periods (30d) can be prescribed as part of the stimulation program.

Although not shown in FIG. 2, stimulation pulses can also be monophasic, having only a single actively driven phase (30a). Because monophasic pulses lack an active charge recovery phase (30b), such monophasic pulses would typically be followed by passive charge recovery (30c) as just described.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 26a in the IPG 10. The external controller 60 can also have a far-field RF antenna 64b capable of wirelessly communicating with the RF antenna 26b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient's IPG 10 includes a coil antenna 26a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient's IPG 10. If the IPG 10 includes an RF antenna 26b, the wand 76, the computing device, or both, can likewise include an RF antenna 74b to establish communication with the IPG 10 at larger distances. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

FIG. 5A shows GUI 99 as may be rendered on an external system to program the stimulation the IPG 10 provides. The GUI 99 includes a waveform interface 100 which allows certain stimulation parameters (amplitude I, pulse width PW, and frequency F) to be set or adjusted. Although not shown, waveform interface 100 can include options to set other parameters for the stimulation waveform, like whether biphasic or monophasic pulses are used, whether active and/or passive recovery is to be used, etc. GUI 99 also includes an electrode configuration interface 102 which allows for the selection of electrodes 16 in the electrode array 17 that will provide the stimulation. Interface 102 as shown allows a user to select whether an electrode will operate as an anode, a cathode, or be off (inactive). Further, the percentage of the amplitude (X %) at each active electrode can be specified. In the example shown, electrodes E2, E12, and E13 have been selected to act as anodes, with these electrodes receiving 70, 15, and 15% of the amplitude I respectively as an anodic current. That is, E2 will provide 0.7*+I, while E12 and E13 each provide 0.15*+I. Electrodes E3, E4, and E14 have been selected to act as cathodes, with these electrodes receiving 40, 40, and 20% of the amplitude I respectively as a cathodic current. That is, E3 and E4 will each provide 0.4*−I, while E14 will provide 0.2*−I. Examples of these waveforms and their relative amplitudes are shown in FIG. 5B, and are shown using biphasic pulses with a first phase 30a having the polarity specified in interface 102 and a second phase 30b of opposite polarity.

GUI 99 in this example also includes a visualization interface 104. Preferably, this interface 104 shows the positioning of leads 15 in the electrode array 17 relative to each other as they are implanted in the patient, and relative to certain tissue structures in the patient such as various vertebrae Vi. These vertebrae could be cervical (C), thoracic (T) lumbar (L), or sacral(S) vertebrae depending where the leads 15 have been implanted in the patient. Other relevant tissue structures could be shown in interface 104 as well. The tissue structures as shown in visualization interface 104 preferably comes from imaging information (e.g., fluoroscopy) taken from the patient.

The visualization interface 104 can also preferably show some indication of the stimulation being provided that is overlaid over the tissue structure and the lead(s) 15. For example, different shading can be used to show which electrodes have been selected to act as anodes (dark), cathodes (light), or that are off (grey). Furthermore, a position of the poles formed by the active electrodes can also be shown. For example, because electrodes E2, E12 and E13 act as anodes, they establish an anode pole (+) at a position in the electrode array 17 influenced by the magnitudes of the anodic current provided at these electrodes (i.e., in between E12 and E13, but closest to E2 because that electrode provides the largest anodic current). Similarly, because electrodes E3, E4 and E14 act as cathodes, they establish a cathode pole (−) at a position influenced by the magnitudes of the cathodic current provided at these electrodes (i.e., in between E3 and E4, and closer to these electrodes because they provide larger cathodic currents). As this example shows, and as mentioned earlier, a pole can be formed in the electrode array 17 using one or more active electrodes (here, three electrodes are used to make each of the anode pole and the cathode pole). This example also illustrates bipolar stimulation, which involves use of a single anode (+) and cathode (−) pole in the electrode array 17. As mentioned earlier, however, stimulation can also be monopolar or multipolar.

As discussed further in U.S. Pat. No. 10,881,859, an electrode configuration algorithm operable in or with the external system rendering GUI 99 can be used to determine the position of the poles given the selection of the electrodes in the electrode configuration interface 102, and as such can indicate the positions of these poles in the visualization interface 104. This algorithm can also operate in reverse. For example, a user can position the anode and/or cathode poles in the electrode array 17 in the visualization interface 104 (using a mouse cursor for example), with the electrode configuration algorithm then operating in reverse to determine which electrodes should be active and with which polarities and amplitudes in electrode configuration interface 102, to form the poles at the specified positions.

### SUMMARY

A method is disclosed for treating an orthostatic intolerance condition of a patient using a spinal cord stimulator comprising an electrode array having a plurality of electrodes. The method may comprise: (a) determining that a patient has the orthostatic intolerance condition by measuring at least one parameter from the patient; (b) if the measured at least one parameter indicates that the patient has the orthostatic intolerance condition, providing a spinal cord stimulator for the patient by implanting the electrode array within the patient's spinal column; (c) providing non-destructive electrical stimulation via the spinal cord stimulator, wherein the stimulation is provided at at least one of the plurality of electrodes; and (d) verifying that the stimulation treats the orthostatic condition by measuring the at least one parameter.

Various examples of this method are also disclosed. In one example, the at least one parameter comprises a heart rate of the patient. In one example, the at least one parameter comprises a change in the heart rate of the patient. In one example, the heart rate is measured using a heart rate sensor. In one example, the heart rate sensor is applied externally to the patient. In one example, the heart rate sensor is integrated with the spinal cord stimulator. In one example, the at least one parameter further comprises a blood pressure of the patient. In one example, the method further comprises adjusting the stimulation using the measured at least one parameter in step (d). In one example, the method further comprises measuring a position of the patient, and adjusting the stimulation using the measured at least one parameter in step (d) and the measured position. In one example, the method further comprises wirelessly receiving the measured position at the spinal cord stimulator. In one example, the position is measured using at least one sensor of the spinal cord stimulator. In one example, step (a) further comprises changing a position of the patient, wherein the at least one parameter is measured before and after changing the position of the patient. In one example, the position of the patient is changed using a tilt table. In one example, the position and the measured at least one parameter are input using a graphical user interface of an external system in communication with the spinal cord stimulator. In one example, the method further comprises wirelessly receiving the position and the measured at least one parameter at an external system in communication with the spinal cord stimulator. In one example, the stimulation stimulates one or more dorsal horns of the patient's spinal cord. In one example, the electrode array is provided longitudinally within the spinal column to configure the stimulation to affect at least one splanchnic nerve innervating the patient's splanchnic bed. In one example, the stimulation comprises a bipole in the electrode array. In one example, the stimulation comprises a monopole in the electrode array. In one example, the stimulation is not perceptible by the patient. In one example, the stimulation is provided using at least one first bipole during first durations and at least one second bipole during second durations, wherein the first and second durations are interleaved in time. In one example, the electrode array comprises a plurality of electrode leads, wherein a first of the electrode leads is positioned proximate to a first dorsal horn of the patient, and wherein a second of the electrode leads is positioned proximate to a second dorsal horn of the patient. In one example, the orthostatic intolerance condition comprises postural orthostatic tachycardia syndrome.

A system is disclosed for treating an orthostatic intolerance condition of a patient, which may comprise: a spinal cord stimulator comprising an electrode array having a plurality of electrodes implantable within the patient's spinal column, wherein the spinal cord stimulator is configured to provide non-destructive electrical stimulation to the patient via one or more of the electrodes; and an external system, wherein the external system is configured to (a) determine that a patient has the orthostatic intolerance condition by measuring at least one parameter from the patient prior providing the stimulation; and (b) verifying that the stimulation treats the orthostatic condition by measuring the at least one parameter.

Various examples of this system are also disclosed. In one example, the system further comprises a heart rate sensor, wherein the at least one parameter comprises a heart rate of the patient measured by the heart rate sensor. In one example, the at least one parameter comprises a change in the heart rate of the patient. In one example, the heart rate sensor is configured to be applied externally to the patient. In one example, the heart rate sensor is integrated with the spinal cord stimulator. In one example, the system further comprises a blood pressure sensor, wherein the at least one parameter further comprises a blood pressure of the patient measured by the blood pressure sensor. In one example, the spinal cord stimulator is further configured to adjust the stimulation using the measured at least one parameter in step (b). In one example, the system further comprises a position sensor configured to measure a position of the patient, wherein the spinal cord stimulator is further configured to adjust the stimulation using the measured at least one parameter in step (b) and the position measured by the position sensor. In one example, the spinal cord stimulator is configured to wirelessly receive the measured position. In one example, the position sensor is within the spinal cord stimulator. In one example, the external system is further configured to change a position of the patient, wherein the at least one parameter is measured in step (a) before and after changing the position of the patient. In one example, the external system comprises a tilt table to change the position of the patient. In one example, the external system comprises a graphical user interface configured to receive the position and the measured at least one parameter. In one example, the external system is configured to wirelessly receive the position and the measured at least one parameter. In one example, the spinal cord stimulator is configured to stimulate one or more dorsal horns of the patient's spinal cord. In one example, the electrode array is configured to be provided longitudinally within the spinal column to configure the stimulation to affect at least one splanchnic nerve innervating the patient's splanchnic bed. In one example, the spinal cord stimulator is configured to provide the stimulation as a bipole in the electrode array. In one example, the spinal cord stimulator is configured to provide the stimulation as a monopole in the electrode array. In one example, the spinal cord stimulator is configured to provide the stimulation such that the stimulation is not perceptible by the patient. In one example, the stimulation is provided using at least one first bipole during first durations and at least one second bipole during second durations, wherein the first and second durations are interleaved in time. In one example, the electrode array comprises a plurality of electrode leads, wherein a first of the electrode leads is configured to be positioned proximate to a first dorsal horn of the patient, and wherein a second of the electrode leads is configured to be positioned proximate to a second dorsal horn of the patient. In one example, the orthostatic intolerance condition comprises postural orthostatic tachycardia syndrome.

A method is disclosed for treating an orthostatic intolerance condition of a patient using a spinal cord stimulator comprising an electrode array having a plurality of electrodes implanted within the patient's spinal column. The method may comprise: (a) providing non- destructive electrical stimulation via the spinal cord stimulator, wherein the stimulation is provided at at least one of the plurality of electrodes; (b) measuring a position of the patient and at least one parameter from the patient, wherein the at least one parameter is indicative of the orthostatic intolerance condition; and (c) adjusting at the spinal cord stimulator the stimulation using the measured position and the measured at least one parameter.

Various examples of this method are also disclosed. In one example, the at least one parameter comprises a heart rate of the patient. In one example, the heart rate is measured using a heart rate sensor. In one example, the heart rate sensor is applied externally to the patient. In one example, the heart rate sensor is integrated with the spinal cord stimulator. In one example, the at least one parameter further comprises a blood pressure of the patient. In one example, the stimulation is adjusted if the measured position indicates that the patient is upright and if the heart rate has increased. In one example, the position of the patient is measured using at least one position sensor. In one example, the at least one sensor comprises an accelerometer. In one example, the accelerometer is within the spinal cord stimulator In one example, steps (b) and (c) are repeated. In one example, the stimulation stimulates one or more dorsal horns of the patient's spinal cord. In one example, the electrode array is provided longitudinally within the spinal column to configure the stimulation to affect at least one splanchnic nerve innervating the patient's splanchnic bed. In one example, the stimulation comprises a bipole in the electrode array. In one example, the stimulation comprises a monopole in the electrode array. In one example, the stimulation is not perceptible by the patient. In one example, the stimulation is provided using at least one first bipole during first durations and at least one second bipole during second durations, wherein the first and second durations are interleaved in time. In one example, the electrode array comprises a plurality of electrode leads, wherein a first of the electrode leads is positioned proximate to a first dorsal horn of the patient, and wherein a second of the electrode leads is positioned proximate to a second dorsal horn of the patient. In one example, the method further comprises wirelessly receiving the measured position and the measured at least one parameter at the spinal cord stimulator. In one example, at least one of the measured position and the measured at least one parameter is input using a graphical user interface of an external system in communication with the spinal cord stimulator. In one example, the method further comprises wirelessly receiving the measured position and the measured at least one parameter at an external system in communication with the spinal cord stimulator. In one example, the orthostatic intolerance condition comprises postural orthostatic tachycardia syndrome.

A system is disclosed for treating an orthostatic intolerance condition of a patient using a spinal cord stimulator comprising an electrode array having a plurality of electrodes configured for implantation within the patient's spinal column. The system may comprise: control circuitry configured to cause non-destructive electrical stimulation via the spinal cord stimulator, wherein the stimulation is provided at at least one of the plurality of electrodes; and a sensor system comprising one or more sensors, wherein the sensor system is configured to measure a position of the patient and at least one parameter from the patient, wherein the at least one parameter is indicative of the orthostatic intolerance condition; and wherein the control circuitry is further configured to adjust at the spinal cord stimulator the stimulation using the measured position and the measured at least one parameter.

Various examples of this system are also disclosed. In one example, the system further comprises the spinal cord stimulator, wherein the control circuitry is within the spinal cord stimulator. In one example, one of the one or more sensors comprises a heart rate sensor, wherein the at least one parameter comprises a heart rate of the patient. In one example, the heart rate sensor is configured to be applied externally to the patient. In one example, the heart rate sensor is integrated with the spinal cord stimulator. In one example, one of the one or more sensors comprises a blood pressure sensor, wherein the at least one parameter further comprises a blood pressure of the patient. In one example, the control circuitry is configured to adjust the stimulation if the measured position indicates that the patient is upright and if the heart rate has increased. In one example, one of the one or more sensors comprises a position sensor for measuring the position. In one example, the at least one sensor comprises an accelerometer. In one example, the accelerometer is within the spinal cord stimulator. In one example, the sensor system is configured to measure the position and the at least one parameter periodically, and wherein the control circuitry is configured to adjust the stimulation in a closed loop fashion using the periodically-measured position and the at least one parameter. In one example, the electrode array is configured to be implanted longitudinally within the spinal column to configure the stimulation to affect at least one splanchnic nerve innervating the patient's splanchnic bed. In one example, the stimulation comprises a bipole in the electrode array. In one example, the stimulation comprises a monopole in the electrode array. In one example, the control circuitry is further configured to cause the stimulation such that the stimulation is not perceptible by the patient. In one example, the stimulation comprises at least one first bipole during first durations and at least one second bipole during second durations, wherein the control circuitry is configured to interleave the first and second durations in time. In one example, the electrode array comprises a plurality of electrode leads, wherein a first of the electrode leads is configured to be positioned proximate to a first dorsal horn of the patient, and wherein a second of the electrode leads is configured to be positioned proximate to a second dorsal horn of the patient. In one example, the control circuitry is configured to wirelessly receive at least one of the measured position and the measured at least one parameter. In one example, the system further comprises an external system configured for communication with the spinal cord stimulator, wherein the control circuitry is within the external system. In one example, the external system comprises a graphical user interface comprising an input for at least one of the measured position and the measured at least one parameter. In one example, the external system is configured to wirelessly receive at least one of the measured position and the measured at least one parameter. In one example, the orthostatic intolerance condition comprises postural orthostatic tachycardia syndrome.

A method is disclosed for treating an orthostatic intolerance condition of a patient using a spinal cord stimulator comprising an electrode array having a plurality of electrodes implantable within the patient's spinal column. The method may comprises: performing a first orthostatic intolerance test on the patient, wherein during the first orthostatic intolerance test at least one parameter indicative of the patient's orthostatic intolerance condition is measured to establish a baseline for the at least one parameter, wherein during the first orthostatic intolerance test either the electrode array has not yet been implanted within the patient's spinal column or if so implanted the spinal cord stimulator does not provide stimulation; and performing a second orthostatic intolerance test on the patient while providing non-destructive electrical stimulation to the patient via the spinal cord stimulator using the electrode array implanted in the patient's spinal column, wherein the stimulation is provided at at least one of the plurality of electrodes, wherein during the second orthostatic intolerance test the at least one parameter is measured to determine a value for the at least one parameter; and comparing the value for each at least one parameter to the baseline for each at least one parameter to assess the efficacy of the stimulation in treating the patient's orthostatic intolerance condition.

Various examples of this method are also disclosed. In one example, the spinal cord stimulator comprises an implantable pulse generator. In one example, the spinal cord stimulator comprises an external trial stimulator. In one example, the first and second orthostatic intolerance tests comprise tilt table tests that move the patient from a supine position to a generally upright position. In one example, the at least one parameter comprises a difference measured with the patient in the supine position and generally upright position. In one example, the at least one parameter comprises a difference in heart rate. In one example, the at least one parameter comprises a difference in blood pressure. In one example, the at least one parameter comprises a subjective measurement. In one example, comparing the value for each at least one parameter to the baseline for each at least one parameter comprises determining a difference of the value for each at least one parameter to the baseline for each at least one parameter. In one example, the baseline for each at least one parameter and the value for each at least one parameter are input to an external system. In one example, the baseline for each at least one parameter and the value for each at least one parameter are telemetered to an external system. In one example, the comparing the value for each at least one parameter to the baseline for each at least one parameter occurs within the external system. In one example, the stimulation stimulates one or more dorsal horns of the patient's spinal cord. In one example, the electrode array is provided longitudinally within the spinal column to configure the stimulation to affect at least one splanchnic nerve innervating the patient's splanchnic bed. In one example, the stimulation comprises a bipole in the electrode array. In one example, the stimulation is not perceptible by the patient. In one example, the electrode array comprises a plurality of electrode leads, wherein a first of the electrode leads is positioned proximate to a first dorsal horn of the patient, and wherein a second of the electrode leads is positioned proximate to a second dorsal horn of the patient. In one example, the orthostatic intolerance condition comprises postural orthostatic tachycardia syndrome.

A system is disclosed for treating an orthostatic intolerance condition of a patient. The system may comprise: a spinal cord stimulator comprising an electrode array having a plurality of electrodes implantable within the patient's spinal column, wherein the spinal cord stimulator is configured to provide non-destructive electrical stimulation to the patient via one or more of the electrodes; and an external system, wherein the external system is configured to receive at least one parameter indicative of the patient's orthostatic intolerance condition as measured during a first orthostatic intolerance test on the patient to establish a baseline for the at least one parameter, wherein during the first orthostatic intolerance test either the electrode array has not yet been implanted within the patient's spinal column or if so implanted the spinal cord stimulator does not provide the stimulation, receive a value of the at least one parameter as measured during a second orthostatic intolerance test on the patient, wherein during the second orthostatic intolerance the stimulation is provided to the patient via the spinal cord stimulator using the electrode array implanted in the patient's spinal column, and compare the value for each at least one parameter to the baseline for each at least one parameter to assess the efficacy of the stimulation in treating the patient's orthostatic intolerance condition.

Various examples of this system are also disclosed. In one example, the spinal cord stimulator comprises an implantable pulse generator. In one example, the spinal cord stimulator comprises an external trial stimulator. In one example, the first and second orthostatic intolerance tests comprise tilt table tests that move the patient from a supine position to a generally upright position. In one example, the at least one parameter comprises a difference measured with the patient in the supine position and generally upright position. In one example, the at least one parameter comprises a difference in heart rate. In one example, the at least one parameter comprises a difference in blood pressure. In one example, the at least one parameter comprises a subjective measurement. In one example, the external system is configured to compare the value for each at least one parameter to the baseline for each at least one parameter by determining a difference of the value for each at least one parameter to the baseline for each at least one parameter. In one example, the external system is configured to receive the baseline for each at least one parameter and the value for each at least one parameter wirelessly by telemetry. In one example, the spinal cord stimulator is configured to stimulate one or more dorsal horns of the patient's spinal cord. In one example, the electrode array is configured to be provided longitudinally within the spinal column to configure the stimulation to affect at least one splanchnic nerve innervating the patient's splanchnic bed. In one example, the spinal cord stimulator is configured to provide the stimulation as a bipole in the electrode array. In one example, the spinal cord stimulator is configured to provide the stimulation such that the stimulation is not perceptible by the patient. In one example, the electrode array comprises a plurality of electrode leads, wherein a first of the electrode leads is configured to be positioned proximate to a first dorsal horn of the patient, and wherein a second of the electrode leads is configured to be positioned proximate to a second dorsal horn of the patient. In one example, the orthostatic intolerance condition comprises postural orthostatic tachycardia syndrome.

### BRIEF DESCRIPTION OF THE DRAWINGS

FIG. 1 shows an Implantable Pulse Generator (IPG), in accordance with the prior art.

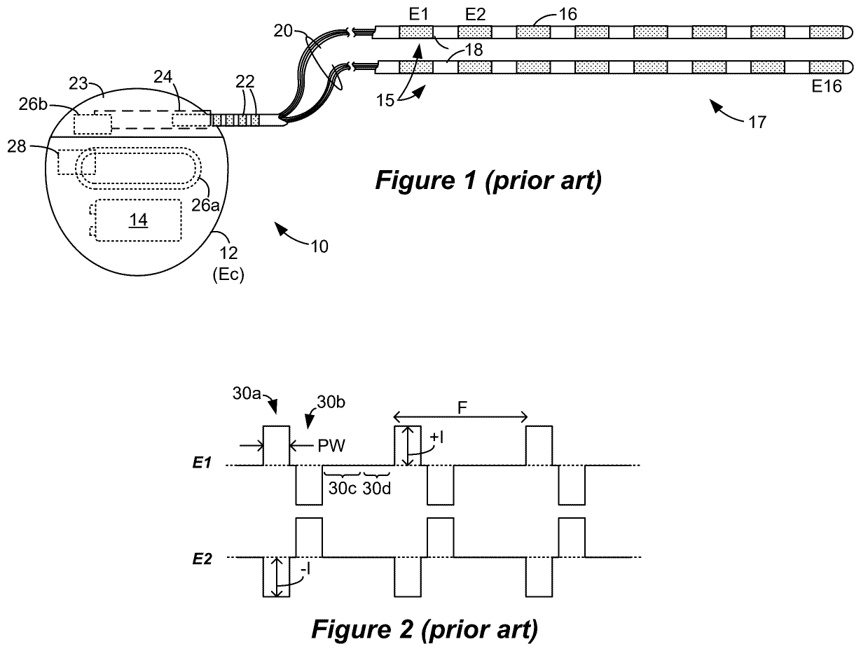

FIG. 2 shows an example of stimulation pulses producible by the IPG, in accordance with the prior art.

FIG. 3 shows stimulation circuitry useable in the IPG, in accordance with the prior art.

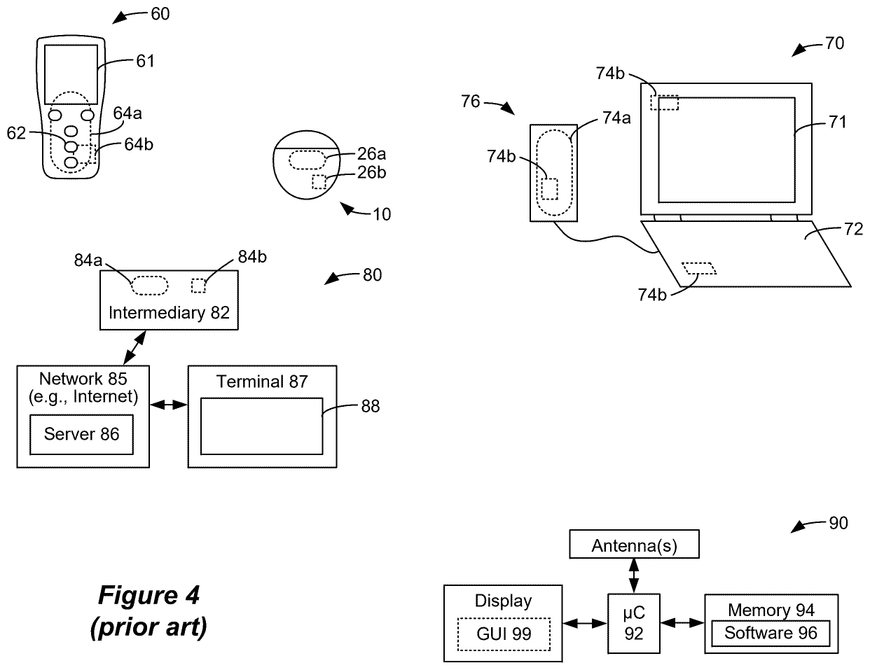

FIG. 4 shows various external devices capable of communicating with and programming stimulation in an IPG, in accordance with the prior art.

FIG. 5A shows a GUI present on an external system to program simulation in an IPG, with FIG. 5B showing an example of waveforms specified by the GUI, in accordance with the prior art.

FIG. 6 shows typical results of a tilt table test performed on a POTS patient.

FIG. 7 shows relevant physiological structures of the spinal cord in the spinal column, and the positioning of the leads relative to these structures in the spinal column, as useful to treating POTS using SCS.

FIG. 8 shows the GUI on an external system used to program simulation in a SCS IPG, and shows preferable stimulation parameters that can be used in treating POTS.

FIG. 9 shows system elements that can be involved in controlling the SCS IPG when treating POTS, including a sensor system capable of wirelessly communicating with an external system and/or the IPG.

FIG. 10 shows the result of a tilt table test performed on a POTS patient both with and without the use of SCS, showing marked improvement in heart rate upon tilting when SCS is applied.

FIG. 11 shows an algorithm for closed loop control of SCS stimulation when treating POTS.

### DETAILED DESCRIPTION

As discussed earlier, Spinal Cord Stimulation (SCS) is typically used to treat symptoms such as back pain. However, the inventors see other uses for SCS, in particular to treat orthostatic intolerance conditions such as Postural Orthostatic Tachycardia Syndrome (POTS). (POTS) is a condition that causes a number of symptoms when a patient transitions from lying/sitting to standing, such as lightheadedness, fainting, dizziness, fatigue, and rapid heartbeat (tachycardia). POTS may also be consequence of a generalized peripheral neuropathy, and other peripheral symptoms may be present that are associated with dysautonomia, such as gastrointestinal, genitourinary, pupillomotor, secretomotor, sleep dysfunction, vasomotor, respiratory, cognitive, vestibular, and sudomotor symptoms, as well as peripheral somatic symptoms such as limb numbness, deficits, and pain. Implementing successful treatments of POTS is particularly needed at present, because evidence suggests that the recent COVID-19 epidemic has caused an increase in POTS cases. See S. R. Raj et al., “Long-COVID Postural Tachycardia Syndrome: an American Autonomic Society Statement,” (published on line Mar. 19, 2021).

POTS is generally understood as a disorder of the autonomic nervous system (dysautonomia), which is largely comprised of the parasympathetic and sympathetic nervous systems. As is known, the sympathetic nervous system is responsible for “fight or flight” responses, while the parasympathetic nervous system acts as a “break” on the sympathetic nervous system. Different subtypes of POTS have been described, such as Neuropathic POTS, Hypovolemic POTS, and Hyperadrenergic POTS (see Mar & Raj, “Postural Orthostatic Tachycardia Syndrome: Mechanisms and New Therapies,” Ann. Rev. Med., 71:235-48 (2020)), based on the hypothesized main cause of the disorder. It is generally understood that in POTS, the sympathetic nervous system ultimately gets dysregulated, thus triggering the tachycardia and postural syndrome.

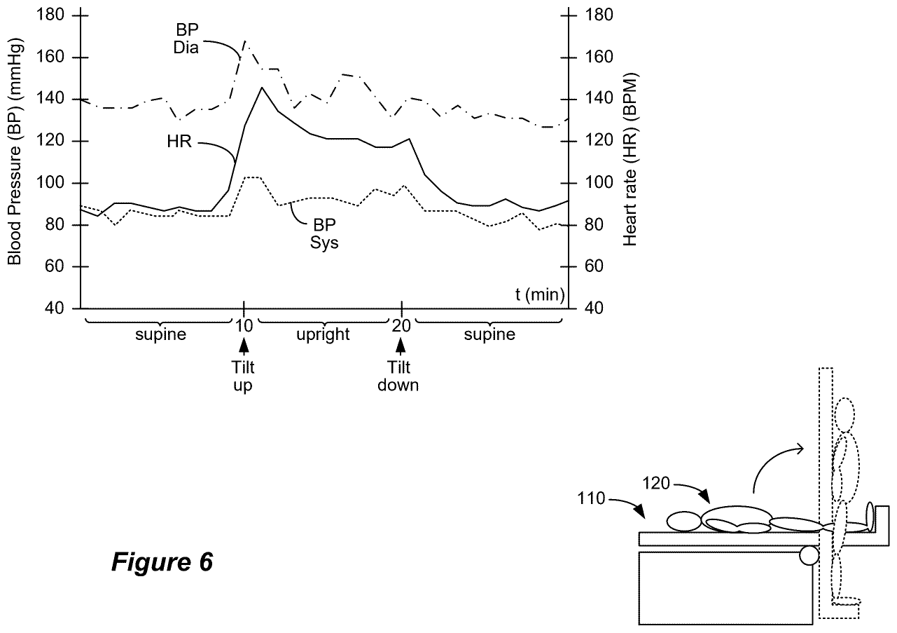

POTS is often diagnosed via a tilt table test. As shown in FIG. 6, a prospective POTS patient 120 is laid in a generally supine position on a tilt table 110, and monitored in that position for some period of time (e.g., 10 minutes) to establish baseline parameters. Such parameters usually include the patient's blood pressure and heart rate as a function of time, which are graphed in FIG. 6. The patient is then tilted to a generally upright position (e.g., at time t=10 minutes). (An upright position may not necessary be a perfectly vertical position). For patients aged 20 or older, POTS may be diagnosed if the patient experiences a sustained heart rate increase of 30 beats per minute or more. Monitoring of blood pressure can be useful to distinguish POTS from other orthostatic intolerance conditions such as orthostatic hypotension, in which the patient experiences a sustained systolic blood pressure drop of 20 mmHg or more upon tilting. The data graphed in FIG. 6 does not show that such a blood pressure drop accompanies the significantly increased heart rate, and so such data generally indicates that the patient suffers from POTS. Later (e.g., at t=20 minutes), the patient is returned to a supine position, causing the increased heart rate to eventually return to normal, again a typical response in POTS patients.

The art has reported that electrical stimulation of the vagus nerve can be helpful in treating POTS symptoms. See P. Chakraborty et al., “Non-invasive Vagus Nerve Simulation in Postural Orthostatic Tachycardia Syndrome,” Arrhythmia Electrophysiol. Rev. 2023; 12: e31 (December 2023). But vagus nerve stimulation, which occurs via the application of electrodes to the vagus nerve outside of the spinal column, only treats parasympathetic responses. Because POTS is believed to have its root cause in dysregulation of the sympathetic nervous system, the inventors hypothesize that the treatment of POTS and other orthostatic intolerance conditions would be better served by spinal cord stimulation (SCS) within the spinal column, which can stimulate and regulate the sympathetic nervous system directly. Further, treating the sympathetic nervous system should help to relieve many of the other somatic and autonomic dysfunctions that that POTS patients suffer (e.g. peripheral sensory symptoms), some of which cannot be treated via parasympathetic. In addition, dorsal column stimulation as may be provided by SCS can also treat other symptoms often present in POTS patients such as sensory deficits and pain caused by dysfunction of peripheral somatic nerves that cannot be modulated by vagus nerve stimulation.

For example, as discussed in U.S. Provisional Patent Application Ser. No. 63/595,616, filed Nov. 2, 2023, it was explained that SCS can regulate the sympathetic nervous system in a manner that affects the allotment of blood capacity in the body. If the sympathetic nervous system is overactive (e.g., in the case of some heart failure patients), SCS can be used to send signals via various splanchnic nerves to the splanchnic bed—certain organs in the abdomen, such as the liver, stomach, spleen, pancreas, and intestines. Together, organs in the splanchnic bed typically hold up to 50% of the body's blood volume. When the sympathetic nervous system is overactive, stimulation of the splanchnic nerves causes blood vessels in the splanchnic bed to constrict. As a result, the splanchnic bed cannot hold as much blood, meaning more peripheral structures in the body will need to carry an excessive blood capacity.

Evidence suggests that POTS patients have significantly impaired sympathetic nervous system activity in the lower extremities. See G. Jacob et al., “The Neuropathic Postural Tachycardia Syndrome,” New England J. of Medicine, Vol. 343, No. 14, pp. 1008-14 (2000). Further evidence suggests that at least some POTS patients experience abnormally increased blood flow and pooling in the splanchnic bed both at rest and in an upright position, and greater decreases in thoracic and cerebral blood flow when compared to healthy controls during tilt testing. See also https://www.hopkinsmedicine.org/health/conditions-and-diseases/postural-orthostatic-tachycardia-syndrome-pots (suggesting that POTS patients can, upon standing or when tilted, either exhibit hypertension (an increase in blood pressure) or hypotension (a decrease in blood pressure)).

Taken together, these observations suggest that improper blood volume allotment may be responsible for symptoms in POTS patients and orthostatic intolerance patients more generally when assuming an upright position. The inventors hypothesize that such dysregulation stems from dysregulation of the sympathetic nervous system, and seek to thus treat POTS and orthostatic intolerance more generally via SCS. The inventors hypothesize that SCS will mediate or normalize operation of the sympathetic nervous system in a manner that would assist orthostatic intolerance patients and POTS patients specifically. For brevity, the below discusses the treatment of POTS via SCS, but again, SCS can be applied to the treatment of orthostatic intolerance more generally.

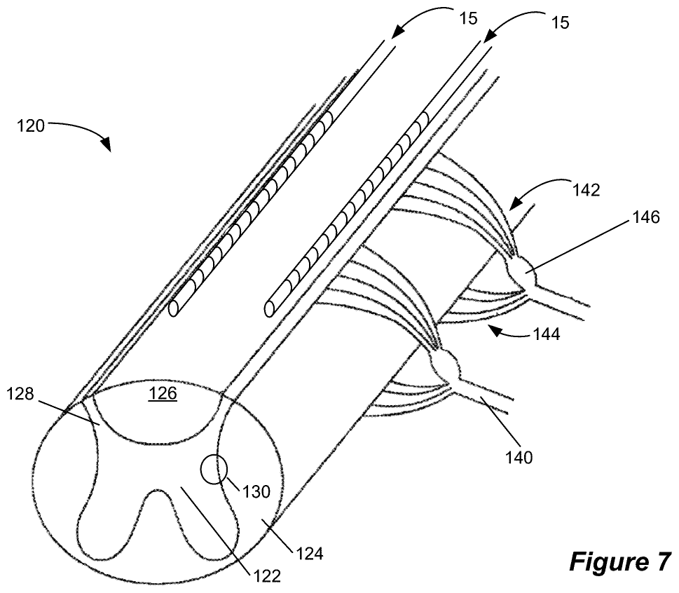

FIG. 7 shows the physiology of the spinal cord 120 within the spinal column, with vertebrae surrounding the spinal cord removed for convenience. A typical transverse section of the spinal cord 120 includes a central “butterfly” shaped central area of grey matter 122 substantially surrounded by an elliptical outer area of white matter 124. The white matter 124 of the dorsal column (DC) 126 includes mostly large myelinated axons that form afferent fibers that run in an longitudinal (rostral/caudal) direction. The dorsal portions of grey matter 122 are referred to as dorsal horns (DH) 128. In contrast to the DC fibers that run in a longitudinal direction, DH fibers can be oriented in many directions, including laterally with respect to the longitudinal axis of the spinal cord. Also shown is the general position of the intermediolateral nucleus (IML) 130 in the grey natter 122. The IML 130 includes sympathetic pre-ganglionic neurons (SPNs) which ultimate innervate various splanchnic nerves as discussed further below.

Also shown in FIG. 7 are spinal nerves 140 that are connected to the spinal cord 120. Spinal nerves 140 are split into a dorsal root (DR) 142 and a ventral root 144, each of which comprise subdivisions referred to as rootlets. The dorsal root 142 also includes a structure called the dorsal root ganglion (DRG) 146, which comprises cell bodies of the afferent neurons. The dorsal roots 142 contains afferent neurons, meaning that they carry sensory signals into the spinal cord, while the ventral roots 144 function as efferent motor roots.

When using spinal cord stimulation to treat conditions impacted by the splanchnic bed such as POTS, it is desirable to non-destructively electrically stimulate the IML 130 (or other grey matter structures coupled to it, like the dorsal horns 128) proximate to spinal nerves 140 that connect to the various splanchnic nerves that innervate the splanchnic bed. As already mentioned, the IML 130 comprises sympathetic pre-ganglionic neurons (SPNs), the modulation of which will modulate operation of one or more the splanchnic nerves. Such splanchnic nerves can include the greater splanchnic nerve connected to spinal nerves 140 proximate to thoracic vertebrae T5-T9; the lesser splanchnic nerve connected to spinal nerves 140 proximate to thoracic vertebrae T10-T11; the least splanchnic nerve connected to spinal nerves 140 proximate to thoracic vertebra T12; and/or the lumbar or sacral splanchnic nerves connected to spinal nerves 140 proximate to lumbar vertebrae L1-L2. To modulate one or more of these splanchnic nerves, the leads 15 in the electrode array 17 should be properly positioned relative to the spinal cord, both longitudinally (i.e., at the correct vertebral level for the splanchnic nerve in question) and laterally such that the leads 15 are proximate to the left and right dorsal horns 128 as shown in FIG. 7. When treating POTS, spinal cord stimulation is preferably applied at least at thoracic levels T6-T12, which modulates activity on the greater, lesser, and least splanchnic nerves. In addition, the inventors hypothesize that stimulation of dorsal columns via SCS at those levels may help to regulate vasodilation/vasodilation of the lower limbs, which has been described as impaired in POTS. See M. Wu et al., “Putative Mechanisms Behind Effects of Spinal Cord Stimulation on Vascular Diseases: A Review of Experimental Studies,” Auton Neurosci. 138(1-2): 9-23 (Feb. 29, 2008).

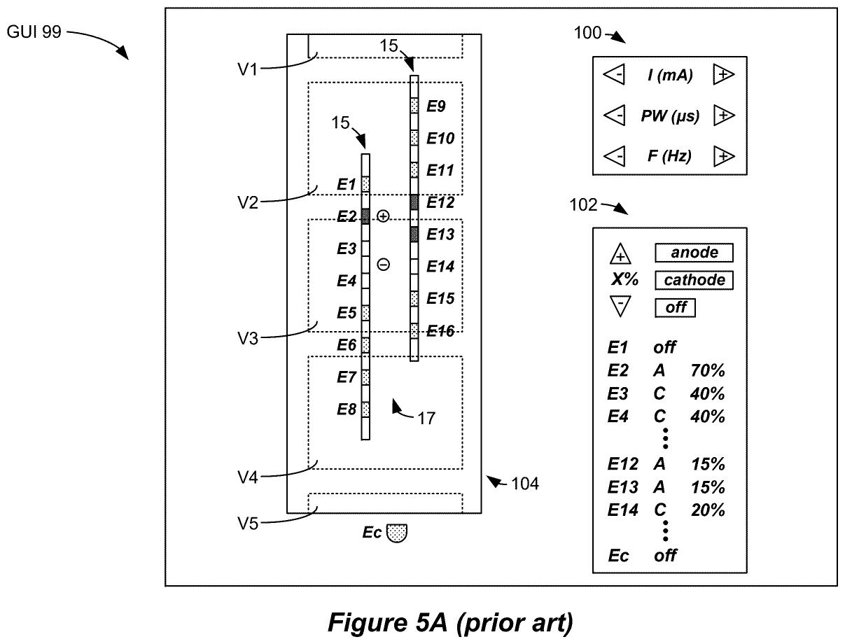

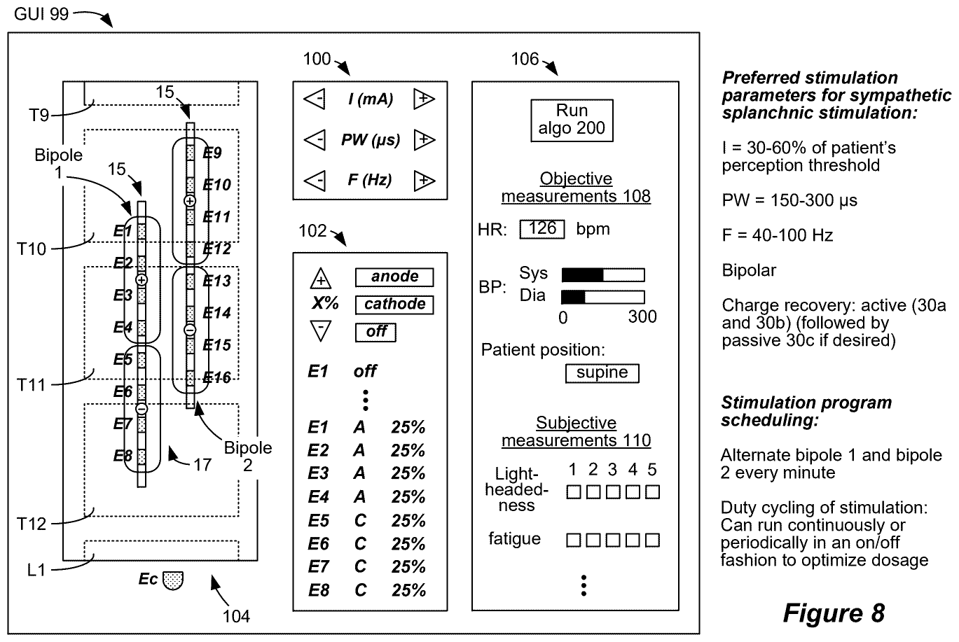

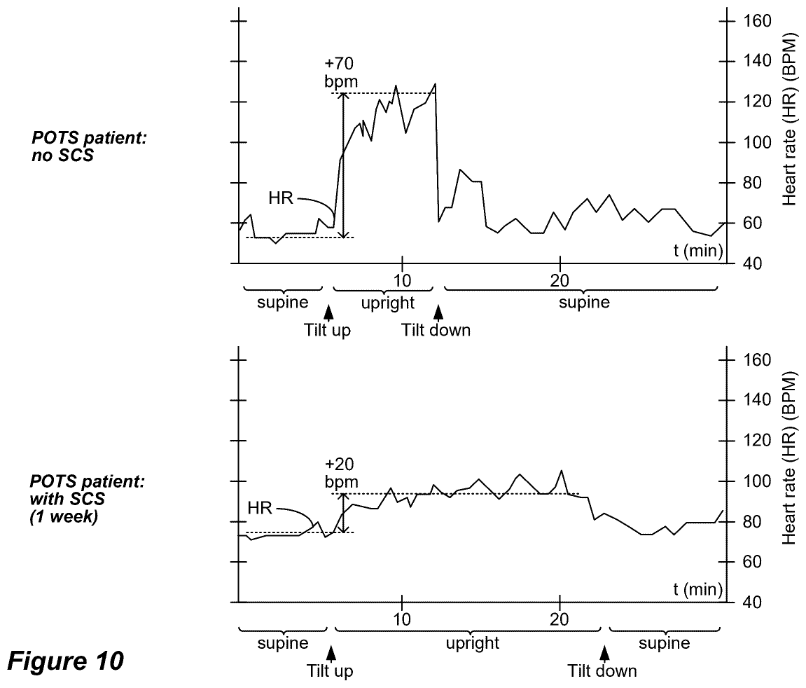

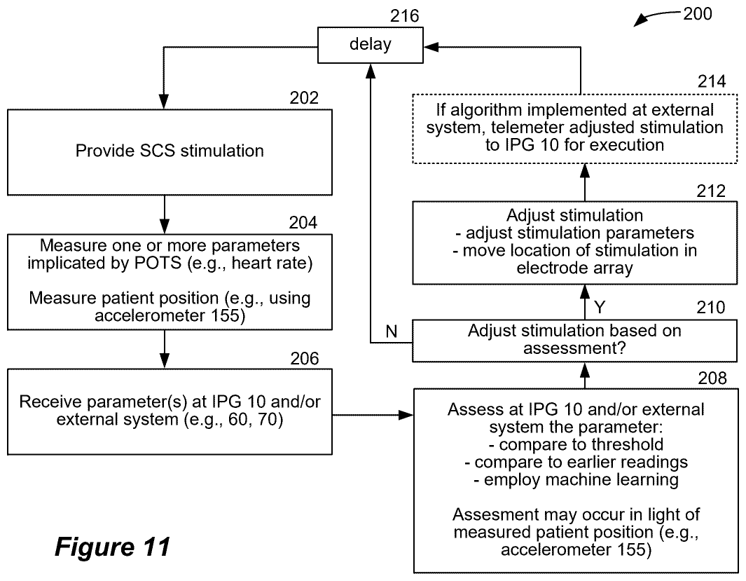

FIG. 8 shows the GUI 99 as used to program the SCS IPG 10, and in particular shows programming useful for treating conditions like POTS. GUI 99 is shown as implemented on an external system, and in particular is as might be present on the clinician programmer 70 (FIG. 4). As such, GUI 99 can be useful to the clinician when fitting the patient, i.e., when setting or adjusting stimulation parameters to best treat a patient's POTS symptoms, or to verify that SCS has had a positive effect on POTS symptoms (as shown later in FIG. 10). However, GUI 99, or portions thereof, may also be present on the patient's external controller 60 (FIG. 4), where it can likewise be used to set or adjust stimulation in a closed loop fashion (as shown later in FIG. 11).

As shown in the visualization interface 104, the leads 15 in the electrode array 17 have been positioned in the spinal column proximate to the T9-T12 vertebrae, which as noted above, are generally proximate to spinal nerves that couple to the greater, lesser, and least splanchnic nerves. The inventor expects that positioning of the leads 15 at these locations would provide the best opportunity to modulate the sympathetic nervous system (e.g., at lower thoracic positions). That being said, the leads 15 could be positioned elsewhere in the spinal column, i.e., proximate to spinal nerves coupled to splanchnic nerves at other longitudinal positions. As noted earlier, the leads 15 are preferably positioned close to the dorsal horns 128 at these longitudinal locations, due to the dorsal horn 128′s connection to the IML 130 that affects the sympathetic nervous system.

When spinal cord stimulation is used to treat pain, it is typically advisable to provide stimulation via an electric field at a concentrated position in the electrode array 17. This is typically provided by bipolar stimulation with an anode and cathode pole in the electrode array 17 that are close together, and at a location that precisely recruits the patient's pain. See, e.g., U.S. Pat. No. 10,576,282. However, when treating conditions like POTS that are impacted by the splanchnic bed, the inventor hypothesizes that a more diffuse electric field can be used that is less targeted to a particular position. Therefore, as shown in FIG. 8, SCS stimulation in this context can employ a bipole that is more diffused and spread in the electrode array 17. For example, a first bipole (bipole 1) can be formed using all of the electrodes on one of the leads 15, with four being used to form the anode pole (E1-E4, each providing 25% of the anodic current, 0.25*+I), and four being to form the cathode pole (E5-E8, each providing 25% of the cathodic current, 0.25*−I), as shown in the electrode configuration interface 102. The current could also be fractionalized in a manner that puts more of the current at the farthest extent of the bipole (e.g., more current at electrodes E1 and E8 compared to electrodes E2 and E7). Examples of how bipole 1 may be formed and configured are disclosed in U.S. Pat. Nos. 10,549,097 and 11,376,433, and U.S. Patent Application Publication 2022/0296902.

Furthermore, when SCS is used to treat POTS, it is preferable that the stimulation provided is not perceptible to the patient. This is in contrast to the use of SCS to treat pain, because in that context the perceived stimulation (paresthesia) can be useful to “cover” the pain that the patient is feeling. However, because pain may not be present when using SCS stimulation to treat POTS, it is not necessary that the patient feel the spinal cord stimulation and experience paresthesia. As such, it is preferable in this context that the stimulation be below the patient's perception threshold. This can occur as follows. Once appropriate stimulation is determined for the patient (e.g., frequency, pulse width, and active electrodes have been set), the amplitude I of the stimulation can be adjusted to be below that which the patient can feel. As noted in FIG. 8, this sub-perception level can be 30 to 60% of the patient's perception threshold—i.e., 30 to 60% of the amplitude I required for the patient to feel the stimulation.

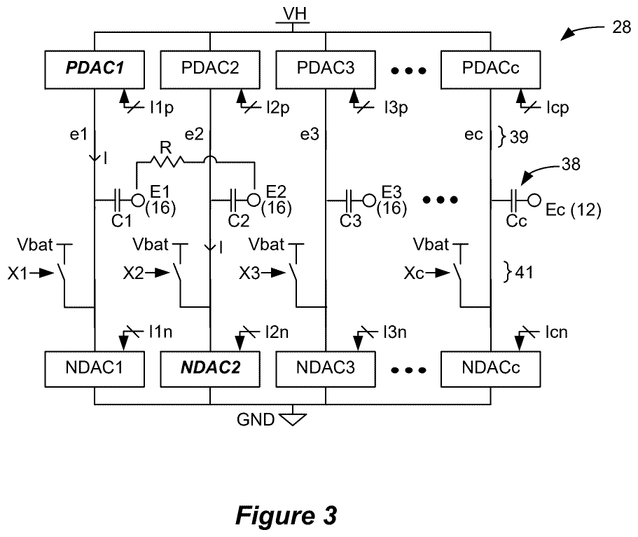

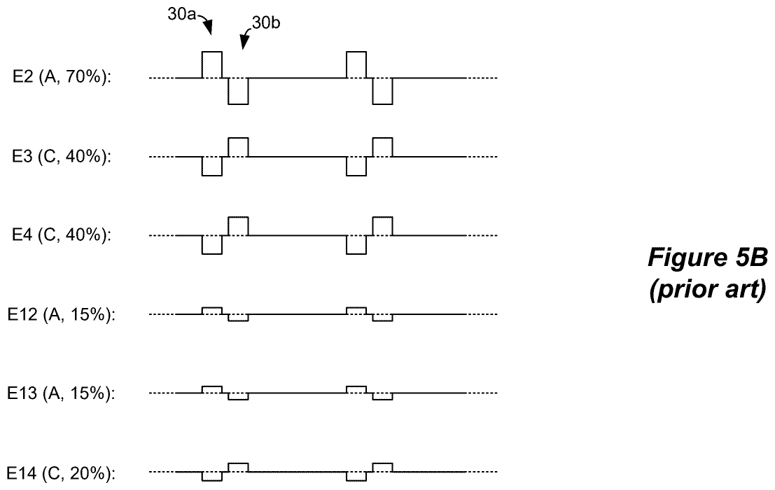

Other stimulation parameters useful when providing SCS to treat POTS include a frequency in the range of 40 to 100 Hz, and a pulse width of 150 to 300 microseconds. Furthermore, it is believed that the provided stimulation pulses should involve the use of active charge recovery—i.e., using an active recharge phase 30b following the first phase pulses at 30a (see FIG. 2). Thus, although not shown in FIG. 8, at bipole 1, the polarity would be flipped in this second phase 30b, with electrodes E1-E4 providing cathodic currents, and E5-E8 providing anodic currents. Reversing the polarity at the active electrodes during an active charge recovery phase 30b was described earlier with respect to FIG. 5B. If necessary or desirable, the active charge recovery phase 30b can be followed by the use of passive charge recovery (30c). Although not shown, the selection of the use of active or passive charge recovery can be provided by the GUI 99. Further examples of stimulation parameters useable in this context, and strategies for selecting such parameters, are disclosed in U.S. Patent Application Publication 2020/0009367.

While use of bipolar stimulation (and in particular use of a diffuse bipole) is expected to provide optimal stimulation when treating POTS, monopolar stimulation may be suitable as well. As discussed above, in monopolar stimulation, the electrode array 17 carries a single anode pole or cathode pole, with the case electrode 12 providing the current return (programmed to provide a pole of the opposite polarity). When using monopolar stimulation, a diffuse pole comprising a plurality of electrodes (e.g., E1-E4, or E5-E8) can be used.

Although the details aren't shown in FIG. 8, the GUI 99 can also be used to schedule the prescribed stimulation. For example, stimulation used to treat POTS can be provided at different locations in the electrode array 17 at different times, and in this regard FIG. 8 shows the use of bipole 1 (E1-E8) and bipole 2 (E9-E16). Because these bipoles are formed at thoracic locations (e.g., T9-T12), they would be effective in stimulating splanchnic nerves, and therefore affect lower thoracic structures like the splanchnic bed, the gastrointestinal and genitourinary tracts, and otherwise treat sensory deficits in the lower limbs. Although not shown, another bipole (bipole 3) could be provided on a different lead implanted at a different position in the spinal column (e.g., C5-T2). At this thoracic location, a bipole would be effective in treating sensory deficits in the upper limbs, which might be indicated for some POTS patients. The various bipoles can be interleaved in time, with each being provided for a certain duration (e.g., a minute or so each). The stimulation provided by different bipoles can be provided in different timing channels in the IPG 10. Stimulation can also be duty cycled, with the stimulation being applied periodically (e.g., ten minutes on), followed by an off period (e.g., ten minutes off). This can be useful in tailoring the dosage of charge or current provided to the patient's tissue.

Also included in the GUI 99 of FIG. 8 is a measurement interface 106. This interface 106 allows for receipt of various measurements relevant to POTS symptoms. For example, interface 106 includes a selectable option to run a closed loop algorithm 200 that adjusts the stimulation based on various measurements indicative of symptoms. This algorithm 200 is explained further below with reference to FIG. 11. Relevant measurements can be objective or subjective, and can be used to determine if the stimulation is having the intended effect in treating POTS symptoms.

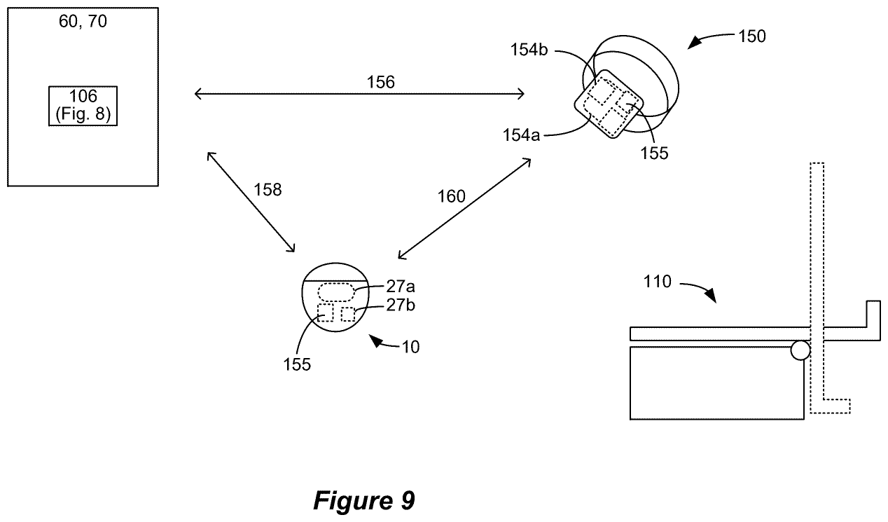

Objective measurements can be input or reviewed at an option 108 in interface 106. As one example of an objective measurement, option 108 shows the heart rate (HR) of the patient. This can be monitored by a sensor system 150, as explained further below with reference to FIG. 9. Option 108 also shows receipt of the blood pressure of the patient, which can also be monitored by the sensor system 150. The sensor system 150 may also measure and/or determine the position or activity of the patient, such as is measured by an accelerometer 155 comprising part of the sensor system 150, as discussed further below. The sensor system 150 may automatically wirelessly report measurements to the external system implementing GUI 99, again as discussed below. Option 108 may also allow these objective measurements to be manually entered by the clinician, which is useful if the sensor system 150 does not automatically report such measurements to the external system.

Option 110 allows subjective measurements to be input in interface 106. Such subjective inputs are not directly and objectively measured, but nevertheless are indicative of a patient's symptoms. Examples of such subjective measurements for a patient suffering from POTS can include light-headedness and fatigue, which may be input on a scale of 1 (best) to 5 (worst).

Measurement interface 106 may likewise be present on the patient's external controller 60 (see FIG. 9). This is useful so that the patient's controller may similarly receive or allow the input of subjective or objective measurements relevant to POTS, and to in turn allow the SCS stimulation parameters to be adjusted once initially fitting has occurred using the clinician programmer 70.

As discussed above, a sensor system 150 can be used to determine if the stimulation is having the intended effect in treating POTS, and an example of a sensor system 150 is shown in FIG. 9. The system shown in FIG. 9 can be used to verify that SCS has been effective in treating a patient's POTS symptoms, for example in conjunction with tilt table testing as discussed below with reference to FIG. 10. This system can also be used to adjust the stimulation a POTS patient's SCS device supplies, as explained further below with reference to FIG. 11.

The sensor system 150 can include one or more sensors relevant to assessing POTS symptoms and the effectiveness of the SCS therapy. The sensor system 150 can be integrated, and in this example the sensor system 150 comprises a combined heart rate and blood pressure sensor, which preferably monitors the patient's heart rate and blood pressure at a peripheral location of the patient. In the example shown, the sensor system 150 is shown as wearable on a patient's wrist or arm, but may take other form factors as well (a patch, a wristwatch, etc.), and may be located at different locations on the patient's body. In other examples, the sensor system 150 may be implantable, and thus able to measure the patient's heart rate or blood pressure internally. Sensor system 150 may also be at least partially integrated with the IPG 10; the IPG 10 could include the heart rate or blood pressure sensors for example. The sensor system 150 can comprise a tissue sensing signal in the IPG 10, and in this regard U.S. Patent Application Publication 2019/0290900, which is incorporated by reference herein, discloses sensing heart rate and other cardiac parameters via a sensed tissue signals in an SCS IPG. Sensors in the sensor system 150 may also measure other cardiovascular parameters such as heart rate variability, O2 saturation, various ECG parameters, parameters indicative of vascular resistance, and other cardiovascular parameters. The sensor system can also measure other non-cardiac parameters.

Another sensor useful in the sensor system 150 is a sensor capable of determining the posture or activity of the patient—i.e., whether the patient is lying or standing. Such a sensor is of obvious benefit when assessing POTS patients, whose symptoms can worsen when standing. In the depicted example, this sensor comprises an accelerometer 155, although other known types of position sensors could be used as well. The accelerometer 155 can be differently located in the system. For example, it may reside external to the patient, and may be integrated with the heart rate and blood pressure sensors, as shown in FIG. 9. Alternatively, the accelerometer 155 may be located in the SCS IPG 10 itself. This alternative is preferred because the position of the IPG 10 (and hence the accelerometer 155 in the IPG) is generally fixed relative to the patient, and thus serves to more reliably indicate the patient's position compared to body wearable sensors whose position relative to the patient are moveable.

Patient position can also be determined by means other than an accelerometer. For example, patient position can also be determined by detecting and measuring Evoked Compound Action Potentials (ECAPs) that issue from the patient's neural tissue when stimulated. For example, U.S. Patent Application Publications 2022/0323764 and 2022/0266027, incorporated herein by reference in its entirety, discuses techniques for determining patient position using detected ECAPs.

Although the sensor system 150 may be at least partially integrated, the sensor system 150 could also comprise different non-integrated systems—e.g., a separate blood pressure monitoring system; an ECG system for determining heart rate; a discrete position sensing system, etc.

The sensor system 150—that is, the one or more sensors in system 150—are preferably enabled for wireless communications, and may include a near-field magnetic-induction coil antenna 154a and/or a far-field RF antenna 154b. This allows the sensor system 150 to communicate its measurements to an external system (such as the clinician programmer 70 or the external controller 60) and/or to the IPG 10 via communication links 156 and 160 respectively. When measurements are communicated with an external system, that system can permit a user to review the measurements, such as by displaying the measurement on a display associated with that system, e.g., in measurement interface 106 of the GUI 99. Measurements may also be logged at the external system so that they may be reviewed (e.g., graphed) as a function of time. This is useful to allow a user or clinician to determine if the SCS stimulation is having its intended effect on POTS symptoms. Note in FIG. 9 that if the accelerometer 155 is within the IPG 10, position measurements can be reported to the external device 60, 70 directly via antennas 27a/27b (link 158), or indirectly via the sensor system 150 (links 160, 156).

FIG. 10 shows the effectiveness of using SCS as described earlier in treating POTS, and in particular to alleviating tachycardia symptoms associated with POTS. Data is shown for a particular female POTS patient that experienced typical POTS symptoms of orthostatic intolerance, tachycardia, and dizziness, as well as other symptoms resulting from peripheral neuropathy, like gastrointestinal and urogenital symptoms, and sensory deficits such as inability to sense heat and cold. The top graph shows the results of a tilt table analysis for this patient prior to her SCS trial. As shown, upon being tilted upwards, the patient experienced a significant (˜+70 bpm) increase in heart rate and concomitant troubling symptoms. As just described, these heart rate measurements can be reported to the GUI 99 of the SCS system (108).

Providing this first orthostatic intolerance test is useful to establish a baseline for at least one parameter (e.g., a difference in heart rate, blood pressure, etc.) indicative of the patient's orthostatic intolerance condition while stimulation is not being provided. As such, this test may be performed before the electrode array has been implanted within the patient's spinal column—i.e., before external trial testing (ETS) when the array has been implanted but the external SCS device has not yet, or before the SCS IPG 10 is fully implanted in the patient. Alternatively, this test may be performed after implantation (of the electrode array 17, the IPG 10, or both), with stimulation turned off such that the external SCS device or the IPG 10 does not provide stimulation.

The bottom graph shows the results of a tilt table analysis for this same patient one week after starting SCS therapy as described earlier. As shown, the patient's heart rate is much improved while receiving SCS therapy: while her heart rate still increased upon tilting, this increase was only about +20 bpm, a significant improvement (a difference of −50 bpm), and a significant enough improvement to no longer meet the diagnostic criteria for POTS. Additionally, the patient also reported significant improvements in her other troubling symptoms. In other words, during this second orthostatic intolerance test, a value for each parameter measured earlier can be determined with their values (e.g., +20 bpm) compared to the baseline (+70 bpm) to assess the efficacy of the stimulation in treating the patient's orthostatic intolerance condition.

Wireless communication also allows the IPG 10 to be controlled by objective or subjective measurements in a closed loop fashion, thus allowing the IPG 10 to adjust the provided stimulation based upon the measurements. FIG. 11 shows a closed loop algorithm 200 useful in this regard. The algorithm 200 can be implemented within the IPG 10, the external system (e.g., external controller 60, or clinician programmer 70), or both, and variations in this regard are discussed further below. The algorithm 200 may include instructions embodied in one or more non-transitory computer readable media, such as solid state, magnetic, or optical memory, which may reside in the external system (94, FIG. 4), or in the IPG 10. The computer readable medium may also reside in a server in communication with the external system and/or the IPG 10, allowing the algorithm 200 to be downloaded into these devices.

As shown in FIG. 11, the closed loop algorithm 200 starts by providing SCS stimulation (202), as discussed above. Next, one or more parameters implicated by POTS are measured (204). As noted earlier, these measurements can be objective (e.g., heart rate as taken by sensor system 150, and received at option 108 of the GUI, FIG. 7) or subjective (e.g., received at option 110). Preferably, the one or more measurements includes a measurement of patient position, such as provided by the accelerometer 150 described earlier. Next, these measured parameter(s) are received at the external system (e.g., 60, 70), the IPG 10, or both (206). Receipt of the measurements can occur at device(s) at which the algorithm 200 has been executed, as explained further below. Such receipt may comprise an input received at option 110 if the measurement is subjective, or may be transmitted by telemetry from the sensor system 150 if objective.

Next, the received measurement(s) are assessed (208), and such assessment can occur in different manners. In one example, each measured parameter can be compared to a threshold, e.g., a heart rate threshold that is indicative of a high rate (tachycardia). The measured parameter can also be compared to earlier measurements to determine, for example, if the patient's heart rate has increased. Machine learning can also be employed to assess the measured parameter(s). As one skilled in the art will understand, such machine learning can iteratively consider measurements and previous stimulation adjustments (discussed next, step 210) to learn how to control operation of the algorithm for best therapeutic results. Received measurement(s) may also be assessed in light of the measured position of the patient, as discussed further below.

Next, the algorithm 200 determines whether to adjust stimulation based on the assessment of the measured parameter made at step 208 (210). Whether adjusting stimulation is warranted can depend on the particular measurement(s) taken and the manner in which that parameter was assessed. For example, if the measured parameter of heart rate is high compared to a threshold, or has been increasing, or has increased sharply, the algorithm 200 may conclude that stimulation adjustment is warranted. As just discussed, machine learning can also determine when it is advisable to adjust the stimulation. Whether adjusting stimulation is warranted can also depend on the position of the patient, as measured at step 204. For example, the algorithm 200 may decide to adjust stimulation if the position measurement indicate that the patient has recently stood up. Still further, the algorithm 200 may decide to adjust stimulation only if position measurements indicate that the patient has recently stood up and the patient's heart rate has simultaneously increased. As noted earlier, the coincidence of these would indicate that the patient is suffering from orthostatic intolerance, making an adjustment to stimulation particularly appropriate.

If the algorithm 200 concludes that the stimulation should be adjusted, various manners in which this adjustment can occur are shown in step 212. Adjustment at this step can involve adjustment of any of the stimulation parameters, including amplitude (I), frequency (F), and/or pulse width (PW). Adjustment can also involve adjustment, use, or non-use of various charge recovery periods (30b, 30c) discussed earlier. Such parameter adjustments can be made to one or more of the poles in the electrodes array 17 (e.g., to bipoles 1 and/or 2). The duration at which the poles are applied may also be adjusted, as may the duty cycle of the stimulation. The location of the stimulation in the electrode array 17 can also be varied at this step.

It may not be known in advance how to adjust the parameters or the location of the stimulation. For example, it may not be known whether to increase or decrease amplitude, or whether it would be better to move the location up/down or right/left in the electrode array 17. Nevertheless, because the algorithm 200 will iteratively adjust the stimulation, such adjustments can be made essentially at random to see which adjustments results in therapeutic improvements. For example, the algorithm 200 may decide to increase the amplitude at this step 212. If this proves to be ineffective based on an assessment of next-received measurements (206, 208), the algorithm 200 could next try decreasing the amplitude. This is true of other stimulation parameters and the location of the stimulation as well, and essentially the algorithm 200 can operate by randomly or pseudo-randomly adjusting the parameters and/or moving the location of the stimulation until better effectiveness (e.g., lower measured heart rates) is achieved. Machine learning, if used, can also eventually learn the best manner to adjust the stimulation at step 212.

Step 214 is an optional step that is particularly useful if the algorithm 200 is implemented in an external system (e.g., 60. 70) as opposed to the IPG 10. After the external system has received and assessed the measured parameter (206, 208), and has determined to adjust the stimulation (210, 212), the external system can telemeter the adjusted stimulation to the IPG 10 along communication link 158 (FIG. 9) to be executed. If the algorithm 200 is instead implemented in the IPG 10, with the IPG 10 receiving and assessing the measured parameter (206, 208), and determining on its own to adjust the stimulation (210, 212), this step 214 is unnecessary.

After the simulation is adjusted (212, 214), or if stimulation does not need to be adjusted (210), the algorithm 200 can impart a delay before repeating (216). This ensures that the algorithm 200 does not react too quickly, or make adjustments to the stimulation too frequently. The delay provided at step 216 can generally range from a second to several minutes.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for treating an orthostatic intolerance condition of a patient using a spinal cord stimulator comprising an electrode array having a plurality of electrodes, the method comprising:
(a) determining that a patient has the orthostatic intolerance condition by measuring at least one parameter from the patient;
(b) if the measured at least one parameter indicates that the patient has the orthostatic intolerance condition, providing a spinal cord stimulator for the patient by implanting the electrode array within the patient's spinal column;
(c) providing non-destructive electrical stimulation via the spinal cord stimulator, wherein the stimulation is provided at at least one of the plurality of electrodes; and
(d) verifying that the stimulation treats the orthostatic condition by measuring the at least one parameter.

2. The method of claim 1, wherein the at least one parameter comprises a heart rate of the patient.

3. The method of claim 2, wherein the at least one parameter comprises a change in the heart rate of the patient.

4. The method of claim 2, wherein the heart rate is measured using a heart rate sensor.

5. The method of claim 4, wherein the heart rate sensor is applied externally to the patient.

6. The method of claim 4, wherein the heart rate sensor is integrated with the spinal cord stimulator.

7. The method of claim 2, wherein the at least one parameter further comprises a blood pressure of the patient.

8. The method of claim 1, further comprising adjusting the stimulation using the measured at least one parameter in step (d).

9. The method of claim 8, further comprising measuring a position of the patient, and adjusting the stimulation using the measured at least one parameter in step (d) and the measured position.

10. The method of claim 9, further comprising wirelessly receiving the measured position at the spinal cord stimulator.

11. The method of claim 9, wherein the position is measured using at least one sensor of the spinal cord stimulator.

12. The method of claim 1, wherein step (a) further comprises changing a position of the patient, wherein the at least one parameter is measured before and after changing the position of the patient.

13. The method of claim 12, wherein the position of the patient is changed using a tilt table.

14. The method of claim 12,
wherein the position and the measured at least one parameter are input using a graphical user interface of an external system in communication with the spinal cord stimulator, or
further comprising wirelessly receiving the position and the measured at least one parameter at an external system in communication with the spinal cord stimulator.

15. The method of claim 1, wherein the stimulation stimulates one or more dorsal horns of the patient's spinal cord.

16. The method of claim 1, wherein the electrode array is provided longitudinally within the spinal column to configure the stimulation to affect at least one splanchnic nerve innervating the patient's splanchnic bed.

17. The method of claim 1, wherein the stimulation comprises a bipole in the electrode array.

18. The method of claim 1, wherein the stimulation is not perceptible by the patient.

19. The method of claim 1, wherein the electrode array comprises a plurality of electrode leads, wherein a first of the electrode leads is positioned proximate to a first dorsal horn of the patient, and wherein a second of the electrode leads is positioned proximate to a second dorsal horn of the patient.

20. A system for treating an orthostatic intolerance condition of a patient, comprising:
a spinal cord stimulator comprising an electrode array having a plurality of electrodes implantable within the patient's spinal column, wherein the spinal cord stimulator is configured to provide non-destructive electrical stimulation to the patient via one or more of the electrodes; and
an external system, wherein the external system is configured to
(a) determine that a patient has the orthostatic intolerance condition by measuring at least one parameter from the patient prior providing the stimulation; and
(b) verifying that the stimulation treats the orthostatic condition by measuring the at least one parameter.
